(19) United States
(12) Patent Application Publication
(10) Pub. No.: US 2019/0322973 A1
(43) Pub. Date: Oct. 24, 2019

(54) CAPTURE DEVICE FOR A CELL PLATE

(71) Applicant: Providence College, Providence, RI (US)

(72) Inventors: Marla Tipping, Hollis, NH (US); James Stephen Waters, East Providence, RI (US)

(21) Appl. No.: 16/392,116

(22) Filed: Apr. 23, 2019

Related U.S. Application Data
(60) Provisional application No. 62/661,916, filed on Apr. 24, 2018.

(51) Int. Cl. C12M 1/12; C12M 3/00; C12M 1/26

(52) U.S. Cl. CPC C12M 25/04; C12M 33/14; C12M 23/48

(57) ABSTRACT

A cell plate includes a well, the well including a capture device, the capture device including a support ring, the support ring having an inner diameter that defines a hollow center and an outer diameter less than an inner diameter of an upper portion of the

[symbol]

### CROSS REFERENCE TO RELATED APPLICATIONS

This application claims benefit from U.S. Provisional Patent Application Ser. No. 62/661,916, filed Apr. 24, 2018, which is incorporated by reference in its entirety.

### STATEMENT REGARDING GOVERNMENT INTEREST

None.

### BACKGROUND OF THE INVENTION

The invention generally relates to metabolic analyzers, and more particularly to a capture device for a cell plate.

In general, various devices, including metabolic analyzers, are employed to measure, track, and analyze data related to cells and tissue which are under analysis. In most cases, the cells or tissue are placed within the wells of a cell plate. The cell plate is then inserted into an analyzer which performs the analysis, displaying the results on a screen or printout for the user.

Analyzing small cells or tissue can be difficult because analyzers often require that the cells or tissue be very near the bottom of the well. However, the cells or tissue often rise within their wells for various reasons, such as the presence of a fluid within the wells causing the cells or tissue to float to the top. This can result in inaccuracies in the analysis or even render the analysis wholly ineffective.

While some apparatus and/or methods have been employed to try to keep cells or tissue within the bottom of the well, these also suffer from a number of deficiencies. For example, uniquely designed tack can be used to affix the contents to the bottom of the well. However, this can often be time consuming for the user. Additionally, the analysis of certain cells requires the analysis to be conducted very soon after dissection. Therefore a process or device which is unable to quickly retain the contents of a well within the bottom of the well can result in further inaccuracies.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a cell plate including a well, the well including a capture device, the capture device including a support ring, the support ring having an inner diameter that defines a hollow center and an outer diameter less than an inner diameter of an upper portion of the well, and a mesh covering affixed to a top surface of the support ring by an adhesive.

In another aspect, the invention features a capture device including a support ring, the support ring having an inner diameter that defines a hollow center and an outer diameter less than an inner diameter of an upper portion of a well within a cell plate of a metabolic analyzer, and a mesh covering affixed to a top surface of the support ring by an adhesive.

In still another aspect, the invention features a method of restraining an article within a well of a cell plate of a metabolic analyzer including forming a support ring having an outer diameter that is less than the inner diameter of the well and an inner diameter defining a hollow center, forming a mesh covering having an outer diameter that is greater than the inner diameter of the support ring, forming a capture device by affixing the mesh covering to the support ring with an adhesive such that the mesh covering extends completely across the hollow center, placing the article within the well, and inserting the capture device above the article within the well such that the capture device runs between interior walls of the well and restrains the article within the well.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 is a front view of an exemplary metabolic analyzer in accordance with the subject disclosure.

FIG. 2 is another front view of an exemplary metabolic analyzer in accordance with the subject disclosure.

FIG. 3 is another front view of an exemplary metabolic analyzer in accordance with the subject disclosure.

FIG. 4 is an overhead view of an exemplary capture device within the well of a cell plate in accordance with the subject disclosure.

FIG. 5 is an overhead view of an exemplary capture device within the well of a cell plate in accordance with the subject disclosure.

FIG. 6 is a chart of results of exemplary experiments related to use of the subject technology in a metabolic analyzer.

FIG. 7 is a flow diagram.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

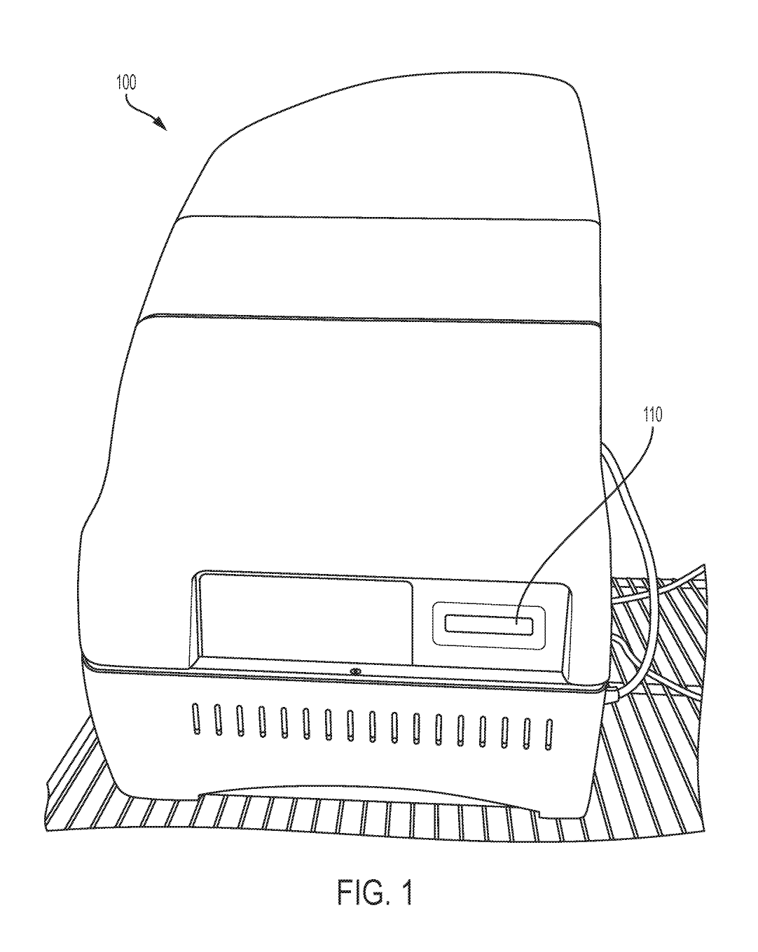

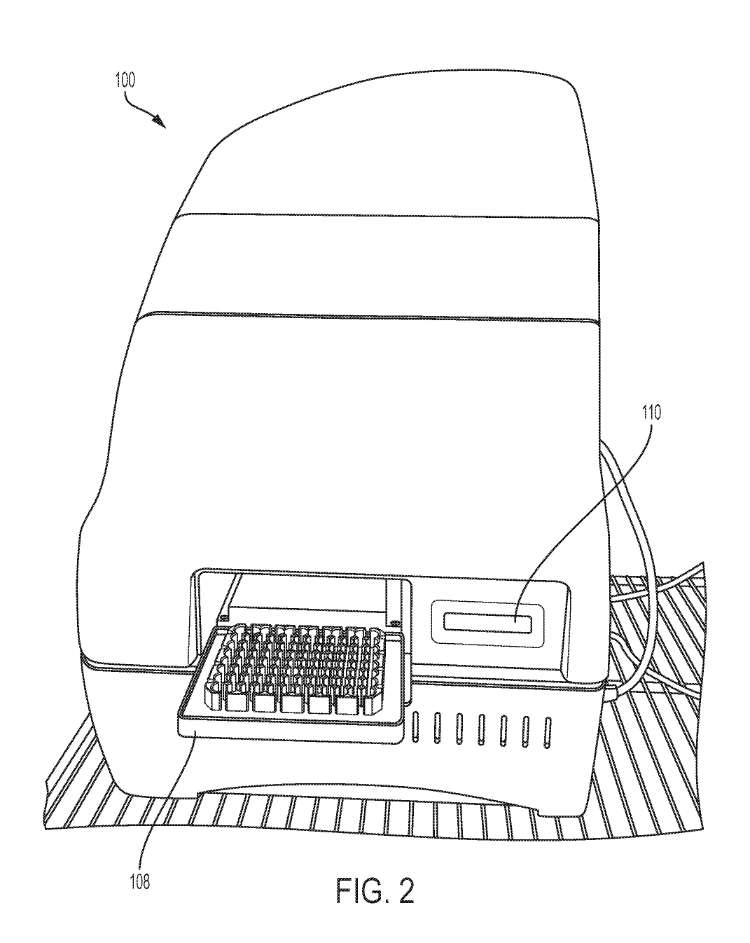

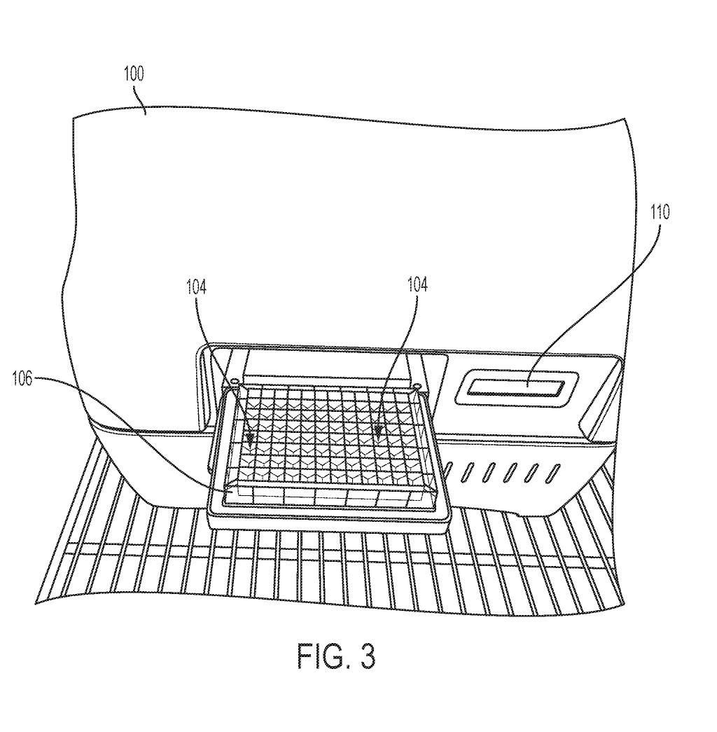

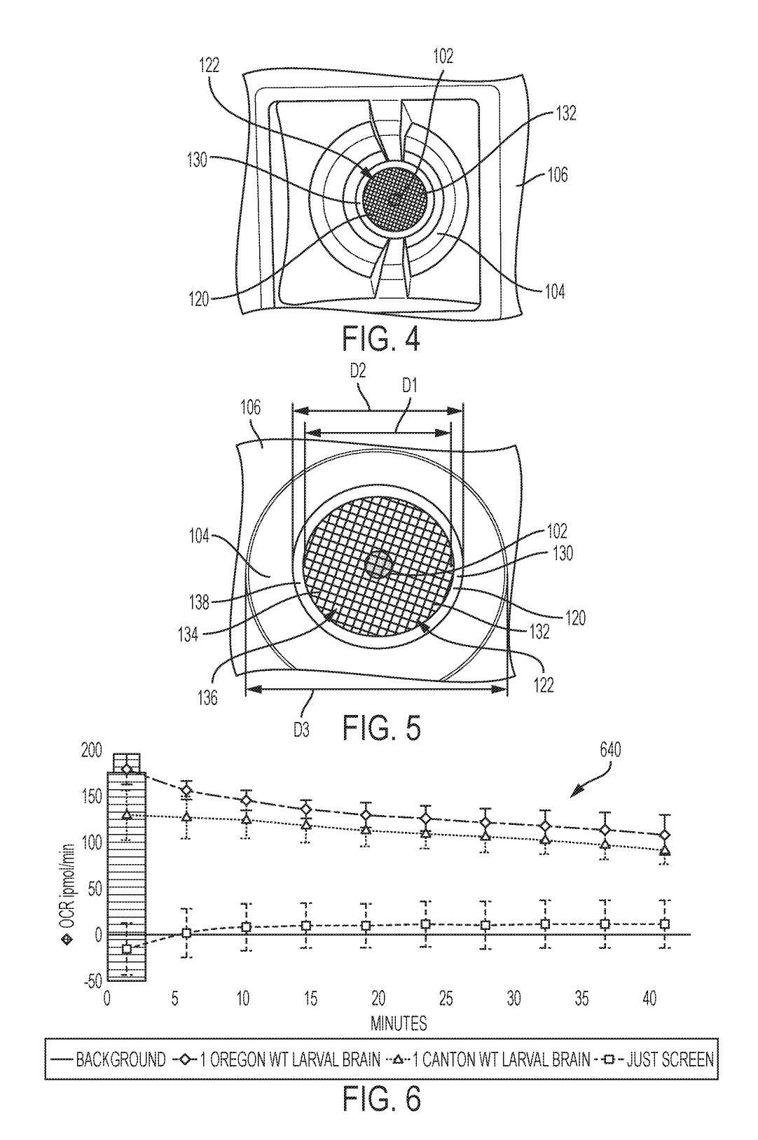

Referring now to FIGS. 1-5, an example of a metabolic analyzer 100 is shown. In the example shown, the metabolic analyzer 100 is an “Agilent Seahorse XFe96” metabolic analyzer sold by Agilent Technologies located at 5301 Stevens Creek Blvd., Santa Clara, Calif. 95051. Notably, this analyzer 100 is very similar to the “Agilent Seahorse XF96” analyzer and the terms “Agilent Seahorse XF96”, “XF96”, or “XFe96” are used throughout the specification to refer to either of the aforementioned analyzers. While the subject technology is not limited to application in any specific analyzer, the subject technology has been found to be effective when implemented with the XF96.

Metabolic analyzers 100, including the XF96, take measurements and generate data with respect to various articles 102. The articles 102 can be, for example, cells, tissue, and/or other organic compounds or the like. The articles 102 are first placed into wells 104 in a cell plate 106. Typically, the cell plate 106 is configured for use with a particular type of metabolic analyzer 100. For example, in the example shown, the cell plate 106 is configured for use with the XF96 analyzer 100. A holding drawer 108 can be extended from the analyzer 100 and the cell plate 106 placed within the holding drawer 108. The holding drawer 108 and cell plate 106 can then be slid into the analyzer 100 to analyze the articles 102 in the capture cells 106.

The analyzer 100 can, for example, measure oxygen consumption rate and extracellular acidification rate of organic material, such as the articles 102 within the cell plate 106. The analyzer 100 is also capable of storing and analyzing data related to the measured data. The analyzer 100 usually includes input devices (such as a screen 110) and output devices (not shown) so a user can effectively operate the analyzer 100 and put the results to use. The subject technology is not limited to any particular analyzer and can be utilized with any analyzer which utilizes a cell plate to measure data related to an article as would be understood by one of skill in the art.

When using a metabolic analyzer 100 it is helpful, and sometimes necessary, for articles 102 being analyzed to be near the bottom of their respective wells 104 within the cell plate 106. The capture device 120 of the subject technology helps improve metabolic analysis, in part, by ensuring that the articles 102 remain close to the bottom of their respective well 104. Notably, while in way of example only one capture device 120 in one well 104 is shown, it is envisioned that the capture device 120 may be use in multiple or all of the wells 104 within a cell plate 106.

As shown, the capture device 120 has a support ring 130. The inner diameter “D1” of the support ring 130 defines a hollow center 122. The outer diameter “D2” of the support ring 130 is less than the inner diameter “D3” of the upper portion of the well 104 such that the support ring 130 can be placed within the well 104. The article 102, a Drosophila brain, for example, sits at the bottom of the well 104 within the hollow center 122 of the support ring 130. The capture device 120 also has a mesh covering 132, formed from crisscrossing wire members 134 which define a number of pores 136, affixed to the top surface 138 of the support ring 130 by an adhesive. The mesh covering 132 extends completely across the hollow center 122 of the support ring 130, securing the Drosophila brain 102 near the bottom of the well 104. Once the capture device 120 is in place, as shown, it will continue to secure the Drosophila brain 102 near the bottom of the well 104 when the cell plate 106 is inserted into a metabolic analyzer 100. Therefore, by keeping the Drosophila brain 102 near the bottom of the well 104, the capture device 120 makes it possible for the metabolic analyzer 100 to produce accurate data related to the Drosophila brain 102.

Various sizes and materials of the components of the capture device 120 have been found to be effective for securing articles 102 and allowing the metabolic analyzer 100 to take accurate readings. For example, some sizes and materials, such as those discussed below in the example embodiments, have been found to be heavy and secure enough to hold the articles 102 in place while not interfering with data collective by the metabolic analyzer 100.

For example, in one embodiment, the support ring 130 is made from a polyoxymetylene material. The mesh covering 132 can be made, for example, from a nylon, or wear-resistant nylon material. The support ring 130 can have an outer diameter D2 of substantially 0.146 inches, an inner diameter D1 of substantially 0.1335 inches, a thickness between the inner and outer diameters D1, D2 of substantially 0.0125 inches, and a height between top 138 and bottom surfaces of the support ring 130 of 0.016 inches. The wire members 134 of the mesh covering 132 can have outer diameters of substantially 0.0018 and define pores 136 can occupy substantially 47% of the surface area defined by the outer diameter of the mesh covering 132. For example, the pores 136 can occupy between 40% and 55% of the surface area of the mesh covering 132. In some cases, the opening size of each pore 136 can be roughly 0.0039 inches. The adhesive used to bond mesh covering 132 with the support ring 130 can be, for example, Loctite 409 made by Henkel Ltd. of Wood Lane End, Hemel Hempstead, Herts HP2 4RQ, United Kingdom. Notably, the dimensions and materials listed above are simply examples that have been found to be effective and are not meant to be all inclusive of all possible dimensions and materials. Further, the dimensions listed are approximate and subject to variations of plus or minus 20 percent.

Referring now to FIG. 6, a chart showing data taken with the analyzer 100 in accordance with the subject technology is shown generally at 640. The chart 640 represents oxygen consumption rate over time with oxygen consumption rate represented on the y-axis and time represented on the x-axis. The line with the lowest oxygen consumption rate (labeled “Just Screen”) represents the effect of placing a cell plate 106 in the analyzer 100 with only the capture device 120 and no underlying article 102 within the cell plate 106. As can be seen, the effect of the capture device 120 is small as compared to the overall oxygen consumption rates of the larval brains 102. Further, the effect of the capture device 120 on oxygen consumption rate is steady and predictable from the 10 minute mark on. Therefore, the effects of the capture device 120 can either be ignored, or factored in and accounted for, depending on the level of accuracy required and/or the goals of the experiment being conducted.

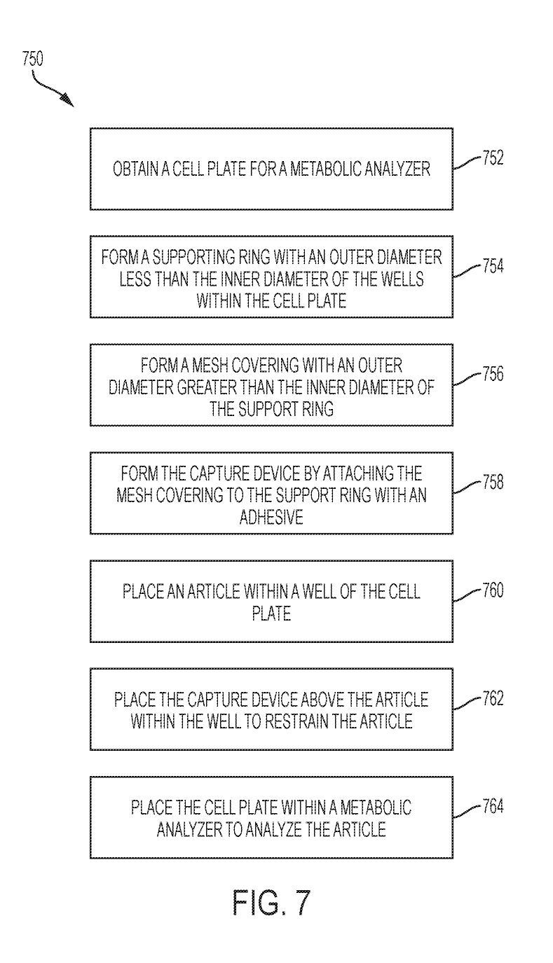

Referring now to FIG. 7, a method 750 for conducting an improved metabolic analysis is shown. Notably, at various times, the method 750 refers to components similar, or the same as, components discussed previously herein. The components utilized in conjunction with the method 750 can be similar to, or the same as, their counterparts discussed above, for example, in terms of material, size, configuration, and function. First, at step 752, a cell plate 106 is obtained. The cell plate 106 is configured for use within a metabolic analyzer 100 and has a number of wells 104 for receiving articles 102 to be analyzed.

At step 754, a support ring 130 is formed. The support ring 130 has an outer diameter D2 that is less that the inner diameter D3 of the wells 104 of the cell plate 106, such that the support ring 130 can fit within the wells 102. The support ring 130 is formed with an annular shape such that an inner diameter D1 of the support ring 130 defines a hollow center 122. In one embodiment, forming the support ring 130 can be accomplished through use of a precision lathe with a cutoff tool. A mesh covering 132 is then formed at step 756. The mesh covering 132 can be formed by crisscrossing wire members 134 and can be circular in shape, such that it has an outer diameter. The outer diameter of the mesh covering 132 is greater than the inner diameter D1 of the support ring 130 such that when the mesh covering 132 is placed over the support ring 130 it completely covers the hollow center 122. In some embodiments, the mesh covering 132 can be formed with an outer diameter that is substantially equal to the outer diameter D2 of the support ring 130 to maximize the surface area contact between the mesh covering 132 and the support ring 130.

A capture device 120 is formed at step 758. To form the capture device 120, the mesh covering 132 is placed over support ring 130 to completely cover the hollow center 122. The mesh covering 132 is affixed to the support ring 130 by an adhesive such that the mesh covering 132 is firmly held in position across the entire hollow center 122. The capture device 120 is then ready for use. Step 758 can be repeated to create multiple capture devices 120 within the various wells 104 of a single cell plate 106.

Next, at step 760, an article 102, for example a piece of tissue, is placed within a well 104 of the cell plate 106 for analysis within a metabolic analyzer 100. This can be carried out multiple times for the various articles 102 and wells 104 within the cell plate 106. For example, this step can be carried out until all of the wells 104 in the cell plate 106 have been filled with articles 102 for analysis. At step 762 capture devices 120 (or a single capture device 120 if only one is needed) are then placed within the wells 104 above the articles 102 to restrain the articles 102 near the bottom of the wells 104. Each capture device 120 runs between the interior walls of a well 104 to prevent the article 102 there within from rising within the well 104. This allows the cell plate 106 to be placed within a metabolic analyzer 100 and the article 102 within the cell plate 106 to be analyzed.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A cell plate comprising:
a well, the well including a capture device comprising:
support ring, the support ring having an inner diameter that defines a hollow center and an outer diameter less than an inner diameter of an upper portion of the well; and
a mesh covering affixed to a top surface of the support ring by an adhesive.

2. The cell plate of claim 1 wherein the mesh covering comprises crisscrossing wire members that define pores.

3. The cell plate of claim 2 wherein the mesh covering is configured to extend across the hollow center of the support ring.

4. The cell plate of claim 1 wherein the support ring is made from a polyoxymetylene material.

5. The cell plate of claim 1 wherein the mesh covering is made nylon.

6. The cell plate of claim 1 wherein the adhesive is a transparent, colorless, ethyl-based instant gel adhesive.

7. A capture device comprising:
a support ring, the support ring having an inner diameter that defines a hollow center and an outer diameter less than an inner diameter of an upper portion of a well within a cell plate of a metabolic analyzer; and
a mesh covering affixed to a top surface of the support ring by an adhesive.

8. The capture device of claim 7 wherein the mesh covering comprises crisscrossing wire members that define pores.

9. The capture device of claim 8 wherein the mesh covering is configured to extend across the hollow center of the support ring.

10. The capture device of claim 7 wherein the support ring is made from a polyoxymetylene material.

11. The capture device of claim 7 wherein the mesh covering is made nylon.

12. The capture device claim 7 wherein the adhesive is a transparent, colorless, ethyl-based instant gel adhesive.

13. A method of restraining an article within a well of a cell plate of a metabolic analyzer comprising:
forming a support ring having an outer diameter that is less than the inner diameter of the well and an inner diameter defining a hollow center;
forming a mesh covering having an outer diameter that is greater than the inner diameter of the support ring;
forming a capture device by affixing the mesh covering to the support ring with an adhesive such that the mesh covering extends completely across the hollow center;
placing the article within the well; and
inserting the capture device above the article within the well such that the capture device runs between interior walls of the well and restrains the article within the well.

14. The method of claim 13 wherein in the step of forming a mesh covering, the outer diameter of the mesh covering is substantially equal to the outer diameter of the support ring.

15. The method of claim 13 wherein, in the step of forming the support ring, the support ring is formed entirely from a polyoxymetylene material.

16. The method of claim 13 wherein, in the step of forming the mesh covering, the mesh covering is formed by crisscrossing wire members which define a plurality of pores through the mesh covering.
